(19) United States
(12) Patent Application Publication
(10) Pub. No.: US 2012/0053413 A1
(43) Pub. Date: Mar. 1, 2012

(54) FLUORESCENT ENDOSCOPY APPARATUS

(75) Inventors: Koji YOSHIDA, Kanagawa-ken (JP); Hitoshi Shimizu, Kanagawa-ken (JP); Masayuki Iwasaka, Kanagawa-ken (JP)

(73) Assignee: FUJIFILM CORPORATION, Tokyo (JP)

(21) Appl. No.: 13/216,181

(22) Filed: Aug. 23, 2011

(30) Foreign Application Priority Data
Aug. 31, 2010 (JP) 193883/2010

(51) Int. Cl. A61B 1/06

(52) U.S. Cl. 600/118

(57) ABSTRACT

A fluorescent endoscopy apparatus includes an endoscope insertion unit that is inserted into a body cavity and that guides excitation light to illuminate a region to be observed, and an imaging unit that images a fluorescent image by receiving fluorescence that has been output from the region to be observed by illumination with the excitation light and guided by the endoscope insertion unit. Further, the fluorescent endoscopy apparatus includes a facial information detection unit that detects facial information about a person in an imaging image that has been imaged by the imaging unit by receiving light guided by the endoscope insertion unit, and an interlock unit that prohibits illumination with the excitation light when the facial information detection unit has detected the facial information.

### BACKGROUND OF THE INVENTION

1. Field of the Invention

The present invention relates to a fluorescent endoscopy apparatus including an endoscope insertion unit that is inserted into a body cavity of a patient . The fluorescent endoscopy apparatus illuminates a region to be observed with excitation light by the endoscope insertion unit, and receives fluorescence output from the region to be observed by the illumination to image a fluorescent image. Especially, the present invention relates to safe control of illumination with the excitation light.

2. Description of the Related Art

Conventionally, endoscope systems for observing tissue in body cavities of patients were widely known. Further, electronic endoscope systems that obtain ordinary images by imaging regions to be observed in body cavities by illumination with white light, and that display the ordinary images on monitor screens, are widely applied to practical use.

As an example of such endoscope systems, an endoscope system that obtains a fluorescent image of ICG (indocyanine green) has been proposed. In the endoscope system, ICG is administered to a region to be observed, in advance, to observe the course of blood vessels and a bloodstream under fat, lymphatic ducts and a lymph stream, the course of bile ducts and a bile stream, and the like, which do not appear in ordinary images. Further, the region to be observed is illuminated with excitation light of near-infrared light to obtain the fluorescent image of ICG.

Since the fluorescence of ICG as described above is weak light, illumination with high-intensity excitation light is necessary to obtain sharper fluorescent images. Therefore, for example, a laser light source of a narrow wavelength band that has a small influence on the contrast between excitation light and fluorescence is used as an excitation light source. However, if high-output-intensity laser light is directly observed by a user or the like, a risk of damaging their eyes exists.

For example, when a procedure of endoscopy ends, and an insertion unit of an endoscope is taken out from a body cavity (in other words, removed from the body cavity and taken out to the outside of the body cavity), if the insertion unit is taken out from the body cavity by mistake without stopping illumination with excitation light, it is dangerous because near-infrared light may enter human eyes.

Therefore, for example, Japanese Unexamined Patent Publication No. 2010-082041 (Patent Document 1) proposes a method for preventing illumination with excitation light. The method utilizes an operation for increasing pressure in an abdominal cavity by using a pneumoperitoneum apparatus during observation by an endoscope. In the method, a pressure sensor is provided at the leading end of an endoscope insertion unit. The pressure sensor detects a change in pressure to detect a state in which the endoscope insertion unit has been taken out from the body cavity. When such a state is detected, illumination with excitation light is not performed,

Further, Japanese Unexamined Patent Publication No. 2002-028125 (Patent Document 2) proposes a method for preventing illumination with excitation light. In the method, a state in which an endoscope insertion unit has been taken out from a body cavity is detected based on the luminance of an image obtained by imaging, the distribution of luminance, color signals and straight line patterns included in the image. When such a state is detected, illumination with excitation light is not performed.

However, if the pressure sensor is provided as disclosed in Patent Document 1, it becomes difficult to reduce the diameter of the leading end of the endoscope. Further, in the method disclosed in Patent Document 2, erroneous detection may occur depending on the surroundings of a place at which the endoscopy apparatus is installed. Therefore, in some cases, it is impossible to prohibit illumination with excitation light in an appropriate manner.

Meanwhile, in recent years, fluorescence of ICG flowing in lymph ducts in a region to be observed was detected by using an endoscope system that obtains a fluorescent image as described above. In the method, ICG is injected into the vicinity of a cancer in advance to check whether the cancer has metastasized to a lymph node. Further, a region to be observed is illuminated with near-infrared light to detect the fluorescence of ICG flowing in lymph ducts.

Further, when a metastasis of a cancer has been found by observing the lymph node using fluorescence as described above, the lymph node is excised and taken out from the body cavity to be provided for pathological examination in some cases.

When the lymph node that has been taken out from the body cavity is provided for pathological examination, it is desirable that a portion of the lymph node to which the cancer has metastasized is cut so that the metastasis portion is easily examined. At this time, a doctor or a user wants to observe a fluorescent image of the lymph node by illuminating the lymph node with near-infrared light outside the body cavity of a patient by using an endoscope system to clearly recognize the metastasis portion in some cases.

However, in the apparatuses disclosed in Patent Documents 1 and 2, if an endoscope insertion unit has been taken out from the body cavity of a patient, it is impossible to perform illumination with excitation light. Therefore, it is impossible to perform observation of a fluorescent image outside the body cavity as described above.

Further, even if observation of a fluorescent image outside the body cavity is possible, it is necessary to prevent entrance of excitation light into human eyes.

### SUMMARY OF THE INVENTION

In view of the foregoing circumstances, it is an object of the present invention to provide a fluorescent endoscopy apparatus that can securely prevent entrance of excitation light into human eyes when an endoscope insertion unit has been taken out from a body cavity, and that can perform observation using fluorescence also outside the body cavity while securely preventing entrance of excitation light into human eyes.

A fluorescent endoscopy apparatus of the present invention is a fluorescent endoscopy apparatus comprising:

an endoscope insertion unit that is inserted into a body cavity and that guides excitation light to illuminate a region to be observed;

an imaging unit that images a fluorescent image by receiving fluorescence that has been output from the region to be observed by illumination with the excitation light and guided by the endoscope insertion unit;

a facial information detection unit that detects facial information about a person in an imaging image that has been imaged by the imaging unit by receiving light guided by the endoscope insertion unit; and

an interlock unit that prohibits illumination with the excitation light when the facial information detection unit has detected the facial information.

The fluorescent endoscopy apparatus of the present invention may further include a canceling-prohibition unit that cancels prohibition of illumination with the excitation light after the illumination has been prohibited by the interlock unit, and an excitation light illumination control unit that checks the result of detecting the facial information from the time when the prohibition of illumination has been canceled by the canceling-prohibition unit, and that controls illumination with the excitation light so that the illumination is not performed while the facial information is being detected by the facial information detection unit.

The fluorescent endoscopy apparatus of the present invention may further include a canceling-prohibition unit that cancels prohibition of illumination with the excitation light after the illumination has been prohibited by the interlock unit, a starting-illumination instruction receiving unit that receives an instruction to start illumination with the excitation light after the prohibition of illumination has been canceled by the canceling-prohibition unit, and an excitation light illumination control unit that checks the result of detecting the facial information from the time when the instruction to start illumination has been received, and that controls illumination with the excitation light so that the illumination is not performed while the facial information is being detected by the facial information detection unit.

A fluorescent endoscopy apparatus according to another aspect of the present invention is a fluorescent endoscopy apparatus comprising:

an endoscope insertion unit that is inserted into a body cavity and that guides excitation light to illuminate a region to be observed;

an imaging unit that images a fluorescent image by receiving fluorescence that has been output from the region to be observed by illumination with the excitation light and guided by the endoscope insertion unit;

a facial information detection unit that detects facial information about a person in an imaging image that has been imaged by the imaging unit by receiving light guided by the endoscope insertion unit; and

an excitation light illumination control unit that controls illumination with the excitation light so that the illumination is not performed while the facial information is being detected by the facial information detection unit.

In a fluorescent endoscopy apparatus of the present invention, the facial information detection unit may detect a round flesh-color portion in the imaging image, and detect the round flesh-color portion as the facial information when the ratio of the area of the round flesh-color portion to the area of the imaging image is greater than or equal to a predetermined threshold value.

According to a fluorescent endoscopy apparatus of the present invention, facial information about a person in an imaging image that has been imaged by an imaging unit is detected. When the facial information has been detected, illumination with excitation light is prohibited, and interlocking is performed. Therefore, it is possible to securely prevent entrance of excitation light into human eyes when an endoscope insertion unit has been taken out from a body cavity.

When a fluorescent endoscopy apparatus of the present invention receives cancellation of prohibition of illumination with excitation light after the illumination has been prohibited, and checks the result of detecting facial information from the time when the prohibition of illumination has been canceled, and does not perform illumination with the excitation light while the facial information is being detected, the following advantageous effects are achievable. Specifically, even after an endoscope insertion unit has been temporarily taken out from a body cavity, it is possible to image, for example, a fluorescent image of a lymph node as described above. Therefore, it is possible to cut a portion of the lymph node in an appropriate manner for pathological examination. Further, it is possible to securely prevent entrance of excitation light into human eyes.

When a fluorescent endoscopy apparatus of the present invention receives cancellation of prohibition of illumination with excitation light after the illumination has been prohibited, and receives an instruction to start illumination with the excitation light after the prohibition of illumination has been canceled, and checks the result of detecting facial information from the time when the instruction to start illumination has been received, and does not perform illumination with the excitation light while the facial information is being detected, advantageous effects similar to the aforementioned advantageous effects are achievable. Specifically, even after an endoscope insertion unit has been temporarily taken out from a body cavity, it is possible to image a fluorescent image. Further, it is possible to securely prevent entrance of excitation light into human eyes.

According to a fluorescent endoscopy apparatus of the present invention, facial information about a person is detected in an imaging image that has been imaged by an imaging unit, and illumination with excitation light is controlled so that the illumination is not performed while the facial information is being detected. Therefore, even when an endoscope insertion unit is taken out from a body cavity by mistake without giving an instruction to stop illumination with excitation light, it is possible to securely prevent entrance of excitation light into human eyes.

When a fluorescent endoscopy apparatus of the present invention detects a round flesh-color portion in the imaging image, and detects the round flesh-color portion as facial information when the ratio of the area of the round flesh-color portion to the area of the imaging image is greater than or equal to a predetermined threshold value, the following advantageous effects are achievable. Specifically, it is possible to indirectly obtain information about a distance between the endoscope insertion unit and a human face by obtaining the ratio of the area of the round flesh-color portion to the area of the imaging image. Therefore, it is possible to securely prevent illumination with excitation light when the endoscope insertion unit is close to the human face. Further, it is possible to prevent erroneous detection of an object other than a human face as facial information. Therefore, it is possible to prevent meaningless prohibition of illumination with excitation light.

### BRIEF DESCRIPTION OF THE DRAWINGS

FIG. 1 is a schematic diagram illustrating the configuration of a rigid endoscope system using a fluorescent endoscopy apparatus according to an embodiment of the present invention;

FIG. 2 is a schematic diagram illustrating the structure of a body cavity insertion unit;

FIG. 3 is a schematic diagram illustrating a leading end of the body cavity insertion unit;

FIG. 4 is a cross section at line 4-4′ in FIG. 3;

FIG. 5 is a diagram illustrating the spectrum of light output from each light projection unit of the endoscope insertion unit, and the spectrum of fluorescence output from a region to be observed by illumination with the light and reflection light;

FIG. 6 is a schematic diagram illustrating the configuration of an imaging unit;

FIG. 7 is a diagram illustrating the spectral sensitivity of the imaging unit;

FIG. 8 is a schematic diagram illustrating the configuration of an image processing apparatus and a light source apparatus; and

FIG. 9 is a flow chart for explaining the action of the rigid endoscope system using a fluorescent endoscopy apparatus according to an embodiment of the present invention.

### DESCRIPTION OF THE PREFERRED EMBODIMENTS

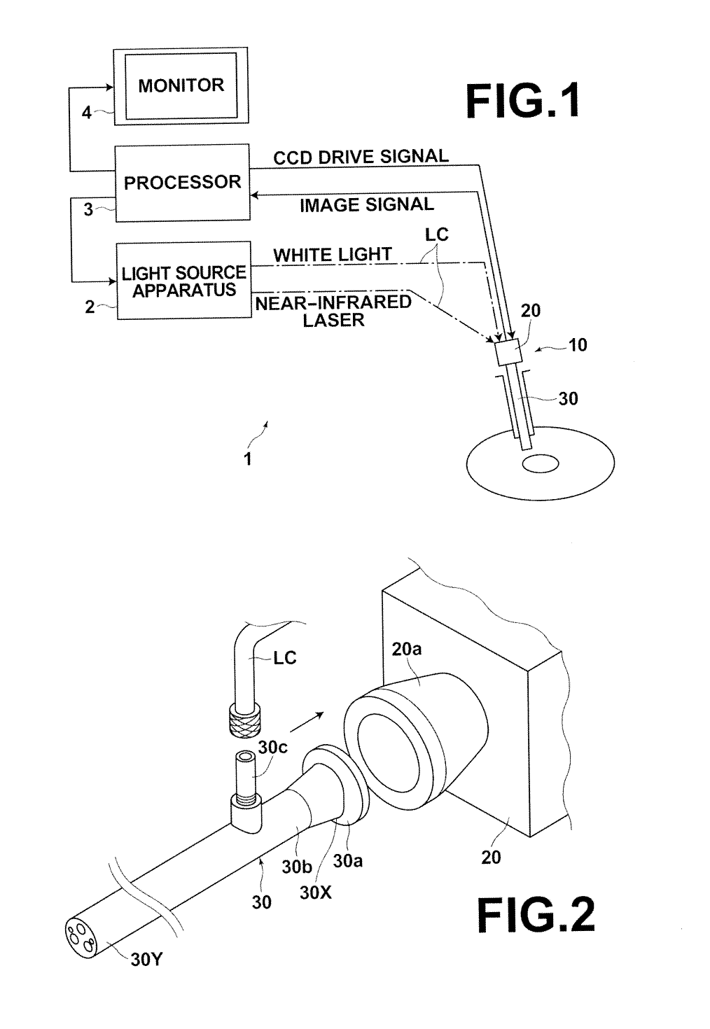

Hereinafter, a rigid endoscope system using a fluorescent endoscopy apparatus according to an embodiment of the present invention will be described in detail with reference to drawings. The fluorescent endoscopy apparatus according to this embodiment controls illumination with excitation light, considering the safety of operations. First, the configuration of the whole system will be described. FIG. 1 is a schematic external view illustrating the configuration of a rigid endoscope system 1 according to the present invention.

As illustrated in FIG. 1, the rigid endoscope system 1 in the present embodiment includes a light source apparatus 2, a rigid endoscope imaging apparatus 10, a processor 3 and a monitor 4. The light source apparatus 2 outputs blue light and near-infrared light. The rigid endoscope imaging apparatus 10 illuminates a region to be observed with white light obtained by performing wavelength conversion on the blue light output from the light source apparatus 2, and the near-infrared light. Further, the rigid endoscope imaging apparatus 10 images an ordinary image based on reflection light reflected from the region to be observed by illumination with the white light, and a fluorescent image based on fluorescence output from the region to be observed by illumination with the near-infrared light. The processor 3 performs predetermined processing on image signals obtained by imaging by the rigid endoscope imaging apparatus 10, and outputs a control signal to the light source apparatus 2. The monitor 4 displays, based on a display control signal generated by the processor 3, a fluorescent image and an ordinary image of the region to be observed.

As illustrated in FIG. 1, the rigid endoscope imaging apparatus 10 includes a body cavity insertion unit 30 and an imaging unit 20. The body cavity insertion unit 30 is inserted into a body cavity, such as an abdominal cavity and a thoracic cavity. The imaging unit 20 images an ordinary image of a region to be observed and a fluorescent image of the region to be observed that have been guided by the body cavity insertion unit 30.

As illustrated in FIG. 2, the body cavity insertion unit 30 and the imaging unit 20 in the rigid endoscope imaging apparatus 10 are connected to each other in a detachable manner. Further, the body cavity insertion unit 30 includes a connection member 30a, an insertion member 30b, and a cable connection opening 30c.

The connection member 30a is provided at an end 30X of the body cavity insertion unit 30 (insertion member 30b). The imaging unit 20 and the body cavity insertion unit 30 are connected to each other in a detachable manner, for example, by fitting the connection member 30a into an opening 20a formed in the imaging unit 20.

The insertion member 30b is inserted into a body cavity when a picture of the inside of the body cavity is taken. The insertion member 30b is made of rigid material, and the shape of the insertion member 30b is, for example, a cylinder with a diameter of approximately 5 mm. Further, a group of lenses for forming an image of the region to be observed is housed in the insertion member 30b. The ordinary image and the fluorescent image of the region to be observed enter the insertion member 30b from a leading end 30Y of the insertion member 30b (body cavity insertion unit 30), and are output from the end 30X of the insertion member 30b (body cavity insertion unit 30) toward the imaging unit 20 through the group of lenses.

The cable connection opening 30c is provided on the side wall of the insertion member 30b, and a light cable LC is mechanically connected to the cable connection opening 30c. Accordingly, the light source apparatus 2 and the insertion member 30b are optically connected to each other through the light cable LC.

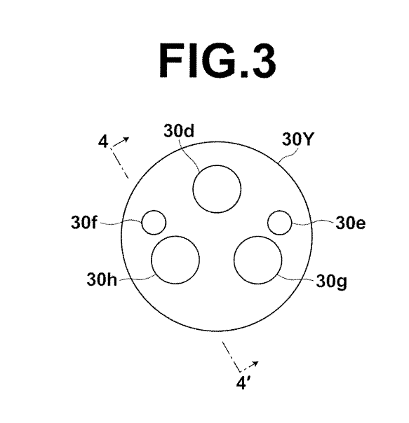

Further, FIG. 3 is a diagram illustrating the structure of the leading end 30Y side of the body cavity insertion unit 30. As illustrated in FIG. 3, an imaging lens 30d for forming an ordinary image and a fluorescent image, illumination lenses 30e, 30f for white light, and illumination lenses 30g, 30h for near-infrared light are provided on the leading end 30Y side of the body cavity insertion unit 30. The illumination lenses 30e, 30f for white light are used to perform illumination with white light, and the illumination lenses 30g, 30h for near-infrared light are used to perform illumination with near-infrared light. The illumination lenses 30e, 30f for white light are arranged substantially symmetrically with respect to the imaging lens 30d. The illumination lenses 30g, 30h for near-infrared light are arranged substantially symmetrically with respect to the imaging lens 30d. The two illumination lenses 30e, 30f for white light are provided and arranged symmetrically, and the two illumination lenses 30g, 30h for near-infrared light are provided and arranged symmetrically, as described above, to prevent generation of a shadow or shadow image in the ordinary image and the fluorescent image by an uneven surface of the region to be observed.

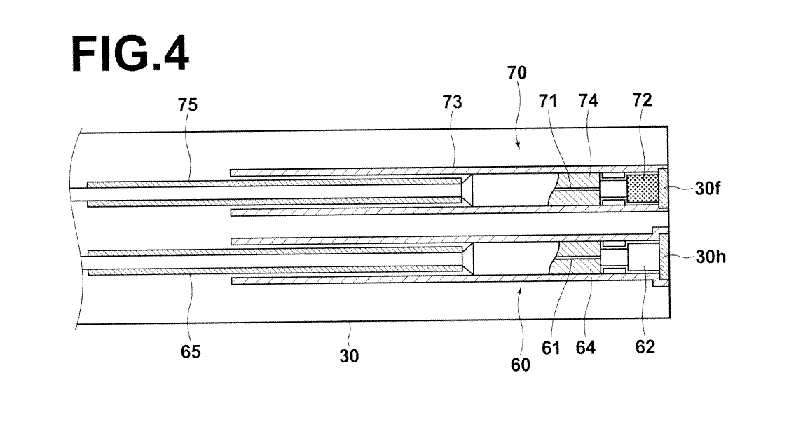

FIG. 4 is a cross section at line 4-4′ in FIG. 3. As illustrated in FIG. 4, a white light projection unit 70 and a near-infrared light projection unit 60 are provided in the body cavity insertion unit 30.

The white light projection unit 70 includes a multi-mode optical fiber 71 for guiding blue light and a phosphor 72. The phosphor 72 is excited by absorption of a part of the blue light guided by the multi-mode optical fiber 71, and outputs visible light of green through yellow. The phosphor 72 is made of a plurality of kinds of fluorescent materials. For example, the phosphor 72 contains fluorescent materials, such as YAG-based phosphor and BAM(BaMgAl₁₀O₁₇) for example.

Further, a cylindrical sleeve member 73 is provided so as to cover the outer circumference of the phosphor 72. Further, a phenyl 74 is inserted into the sleeve member 73 to hold, as a center axis, the multi-mode optical fiber 71. The multi-mode optical fiber 71 extends from the rear end side (which is opposite to the leading end side) of the phenyl 74, and an outer sheath of the multi-mode optical fiber 71 is covered by a flexible sleeve 75 that is inserted between the outer sheath and the sleeve member 73.

Further, the near-infrared light projection unit 60 includes a multi-mode optical fiber 61 for guiding near-infrared light, and a space 62 is provided between the multi-mode optical fiber 61 and the illumination lens 30h for near-infrared light.

Further, a cylindrical sleeve member 63 is provided also in the near-infrared light projection unit 60. The cylindrical sleeve member 63 covers the outer circumference of the space 62. Further, a phenyl 64 and a flexible sleeve 65 are provided in a manner similar to the white light projection unit 70.

As the multi-mode optical fiber used in each of the light projection units, a multi-mode optical fiber with a small diameter may be used. For example, a multi-mode optical fiber with a core diameter of 105 μm, a clad diameter of 125 μm, and a diameter including a protective layer, which forms the sheath of the multi-mode optical fiber, of 0.3 mm to 0.5 mm may be used.

In the above descriptions, the white light projection unit 70 including the illumination lens 30f for white light and the near-infrared light projection unit 60 including the illumination lens 30h for near-infrared light were described. A white light projection unit including the illumination lens 30e for white light and a near-infrared light projection unit including the illumination lens 30g for near-infrared light are structured in a similar manner.

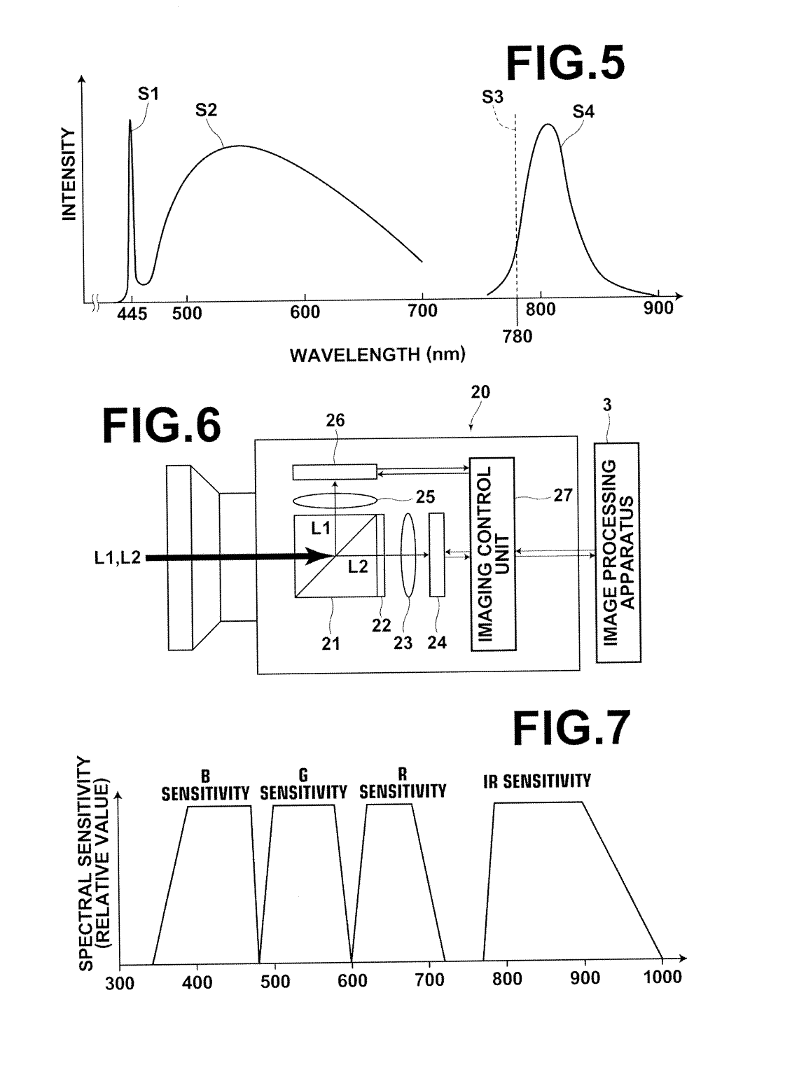

FIG. 5 is a diagram illustrating the spectrum of light output from each light projection unit to illuminate the region to be observed, and the spectrum of fluorescence output from the region to be observed by illumination with the light and reflection light. FIG. 5 illustrates blue light spectrum S1, visible light spectrum S2 of green through yellow, near-infrared light spectrum S3, and ICG fluorescent spectrum S4. The blue light spectrum S1 is the spectrum of blue light that has passed through the phosphor 72 in the white light projection unit 70, and illuminated the region to be observed. The visible light spectrum S2 of green through yellow is the spectrum of visible light of green through yellow that has been excited at the phosphor 72 in the white light projection unit 70, and has illuminated the region to be observed. The near-infrared light spectrum S3 is the spectrum of near-infrared light that has been output by the near-infrared light projection unit 60 to illuminate the region to be observed. The ICG fluorescent spectrum S4 is the spectrum of ICG fluorescence output by illumination with the near-infrared light spectrum S3 by the near-infrared light projection unit 60.

In the specification of the present application, the white light is not limited to light containing strictly all of wavelength components of visible light. It is sufficient if the white light contains light of a specific wavelength band, for example, such as R (red), G (green), and B (blue), which are basic light. Broadly, the white light may be, for example, light containing a wavelength component from green through red, or a wavelength component from blue through green, or the like. Therefore, the blue light spectrum S1 and the visible light spectrum S2 as illustrated in FIG. 5, which are output by the white light projection unit 70 to illuminate the region to be observed, is also regarded as white light.

FIG. 6 is a schematic diagram illustrating the configuration of the imaging unit 20. The imaging unit 20 includes a first imaging system and a second imaging system. The first imaging system generates fluorescent image signals representing the region to be observed by imaging a fluorescent image of the region to be observed that has been formed by a group of lenses in the body cavity insertion unit 30. The second imaging system generates ordinary image signals representing the region to be observed by imaging an ordinary image of the region to be observed that has been formed by a group of lenses in the body cavity insertion unit 30. These imaging systems are separated to two optical axes that are orthogonal to each other by a dichroic prism 21. The dichroic prism 21 has a spectrum characteristic that an ordinary image is reflected and a fluorescent image is transmitted.

The first imaging system includes a near-infrared light cut filter 22, a first image formation optical system 23, and a high-sensitivity imaging device 24. The near-infrared light cut filter 22 transmits a fluorescent image output from the body cavity insertion unit 30, and cuts near-infrared light. The first image formation optical system 23 forms an image of fluorescent image L2 that has been output from the body cavity insertion unit 30 and has passed through the dichroic prism 21 and the near-infrared light cut filter 22. The high-sensitivity imaging device 24 images the fluorescent image L2 formed by the first image formation optical system 23.

The second imaging system includes a second image formation optical system 25 and an imaging device 26. The second image formation optical system 25 forms an image of ordinary image L1 that has been output from the body cavity insertion unit 30 and reflected by the dichroic prism 21. The imaging device 26 images the ordinary image L1 formed by the second image formation optical system 25.

The high-sensitivity imaging device 24 detects light in the wavelength band of the fluorescent image L2 at high sensitivity, and converts the detected light into fluorescent image signals to output the fluorescent image signals. The high-sensitivity imaging device 24 is a monochrome imaging device.

The imaging device 26 detects light in the wavelength band of the ordinary image, and converts the detected light into ordinary image signals to output the ordinary image signals. Color filters of three primary colors of red (R), green (G) and blue (B) are provided on an imaging plane of the imaging device 26 in Bayer arrangement or in honeycomb arrangement.

FIG. 7 is a graph illustrating the spectral sensitivity of the imaging unit 20. Specifically, the imaging unit 20 is structured in such a manner that the first imaging system has IR (near-infrared) sensitivity, and that the second imaging system has R (red) sensitivity, G (green) sensitivity and B (blue) sensitivity.

Further, the imaging unit 20 includes an imaging control unit 27. The imaging control unit 27 performs drive control on the high-sensitivity imaging device 24 and the imaging device 26 based on a CCD (charge-coupled device) drive signal output from the processor 3. Further, the imaging control unit 27 performs CDS/AGC (correlated double sampling/automatic gain control) processing and A/D (analog to digital) conversion processing on the fluorescent image signal output from the high-sensitivity imaging device 24 and the ordinary image signal output from the imaging device 26. The imaging control unit 27 outputs signals after processing to the processor 3 through a cable.

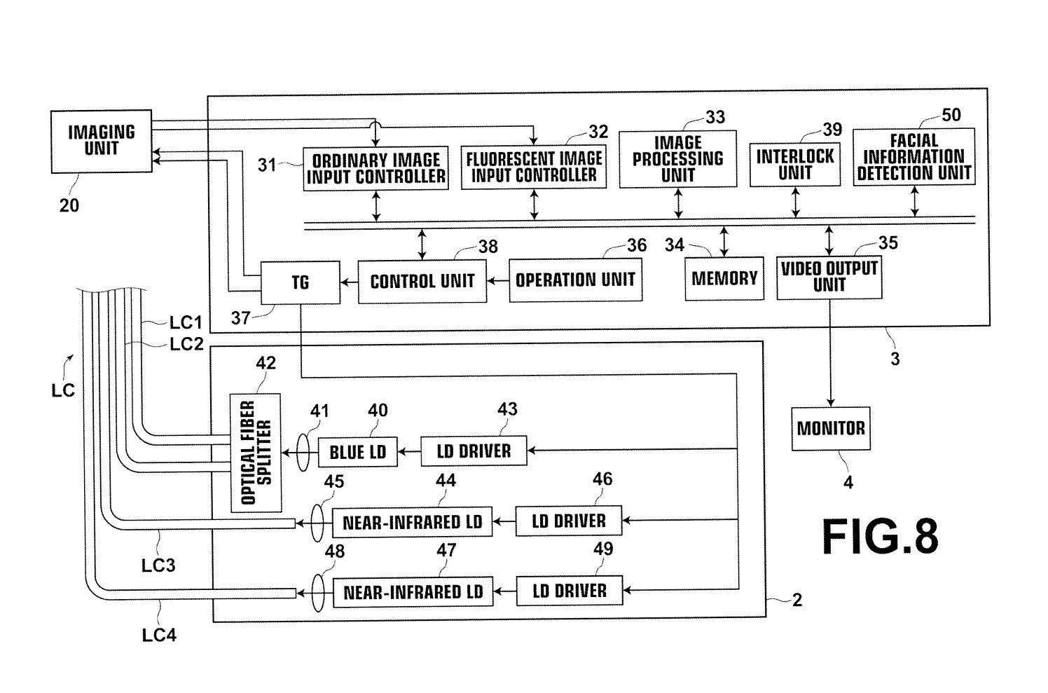

FIG. 8 is a schematic diagram illustrating the configuration of the light source apparatus 2 and the processor 3. As illustrated in FIG. 8, the processor 3 includes an ordinary image input controller 31, a fluorescent image input controller 32, an image processing unit 33, a memory 34, a video output unit 35, an operation unit 36, a TG (timing generator) 37, a control unit 38, an interlock unit 39, and a facial information detection unit 50.

The ordinary image input controller 31 and the fluorescent image input controller 32 include line buffers of predetermined capacities. The ordinary image input controller 31 and the fluorescent image input controller 32 temporarily store ordinary image signals for each frame and fluorescent image signals for each frame, respectively, which have been output from the imaging control unit 27 in the imaging unit 20. The ordinary image signals stored in the ordinary image input controller 31 and the fluorescent image signals stored in the fluorescent image input controller 32 are stored in the memory 34 through buses.

The image processing unit 33 receives ordinary image signals for each frame and fluorescent image signals for each frame that have been read out from the memory 34. The image processing unit 33 performs predetermined image processing on these image signals, and output to a bus.

The video output unit 35 receives, through a bus, the ordinary image signals and the fluorescent image signals output from the image processing unit 33. Further, the video output unit 35 performs predetermined processing on the received signals to generate display control signals. The display control signals are output to the monitor 4.

The operation unit 36 receives an input by an operator or user, such as a predetermined operation instruction and a control parameter.

Especially, the operation unit 36 in the present embodiment receives an instruction to start illumination with near-infrared light and an instruction to release interlock. The instruction to release interlock is input to cancel (release) the state in which illumination with near-infrared light has been prohibited by the interlock unit 39. In the present embodiment, the instruction to start illumination with near-infrared light and the instruction to release interlock are received by the operation unit 36. However, it is not necessary that such instructions are received by the operation unit 36. The instructions may be received, for example, by an operation of pressing a foot pedal or the like.

Further, the TG 37 outputs drive pulse signals to drive the high-sensitivity imaging device 24 and the imaging device 26 in the imaging unit 20, and LD (laser diode) drivers 43, 46, and 49 in the light source apparatus 2, which will be described later.

The control unit 38 controls the whole system. Especially, in the present embodiment, the control unit 38 outputs a control signal to the light source apparatus 2 to stop output of excitation light when the facial information detection unit 50 has detected facial information. A specific method for controlling illumination with excitation light will be described later.

When the facial information detection unit 50 has detected facial information, the interlock unit 39 outputs, based on the detection result, a control signal to the light source apparatus 2 through the control unit 38 to prohibit output of near-infrared light from the light source unit 2. The expression “to prohibit output of near-infrared light” means not only stopping output of near-infrared light, but continuing to prohibit output of near-infrared light until an instruction to cancel prohibition is given, even if an instruction to start illumination with near-infrared light is given.

The facial information detection unit 50 detects facial information in an imaging image based on image signals obtained by imaging by the imaging unit 26 in the imaging unit 20. The facial information detection unit 50 in the present embodiment detects a round flesh-color portion in an imaging image, and detects, as facial information, the round flesh-color portion when the ratio of the area of the round flesh-color portion to the area of the imaging image is greater than or equal to a predetermined threshold value. Since various methods for detecting flesh color are known, detailed descriptions of the methods are omitted. Meanwhile, a round shape should be detected, for example, by detecting a circle, an ellipse, or the like. Since various methods for detecting a round shape are known, detailed descriptions of the methods are omitted. Further, the method for detecting facial information is not limited to the aforementioned methods, and various known methods may be used.

As illustrated in FIG. 8, the light source apparatus 2 includes a blue LD light source 40, a condensing lens 41, an optical fiber splitter 42, and an LD driver 43. The blue LD light source 40 outputs blue light of 445 nm. The condensing lens 41 condenses blue light output from the blue LD light source 40, and makes the condensed light enter the optical fiber splitter 42. The optical fiber splitter 42 makes the blue light, which has entered the optical fiber splitter 42 by the condensing lens 41, enter both of a light cable LC1 and a light cable LC2 simultaneously. The LD driver 43 drives the blue LD light source 40.

Further, the light cables LC1 and LC2 are optically connected to the multi-mode optical fibers 71 in the white light projection units 70, respectively.

The light source apparatus 2 includes plural near-infrared LD light sources 44, 47 that output near-infrared light of 750 to 790 nm, plural condensing lenses 45, 48, and plural LD drivers 46, 49. The plural condensing lenses 45, 48 condense near-infrared light output from each of the near-infrared LD light sources 44, 47, and make the condensed light enter light cables LC3, LC4, respectively. The plural LD drivers 46, 49 drive the near-infrared LD light sources 44, 47, respectively.

The light cables LC3 and LC4 are optically connected to the multi-mode optical fibers 61 in the near-infrared light projection units 60, respectively.

In the present embodiment, near-infrared light is used as excitation light. However, the excitation light is not limited to the near-infrared light. The excitation light may be determined in an appropriate manner based on the kind of a fluorescent dye administered to a patient to be examined or the kind of living tissue the autofluorescence of which is to be induced.

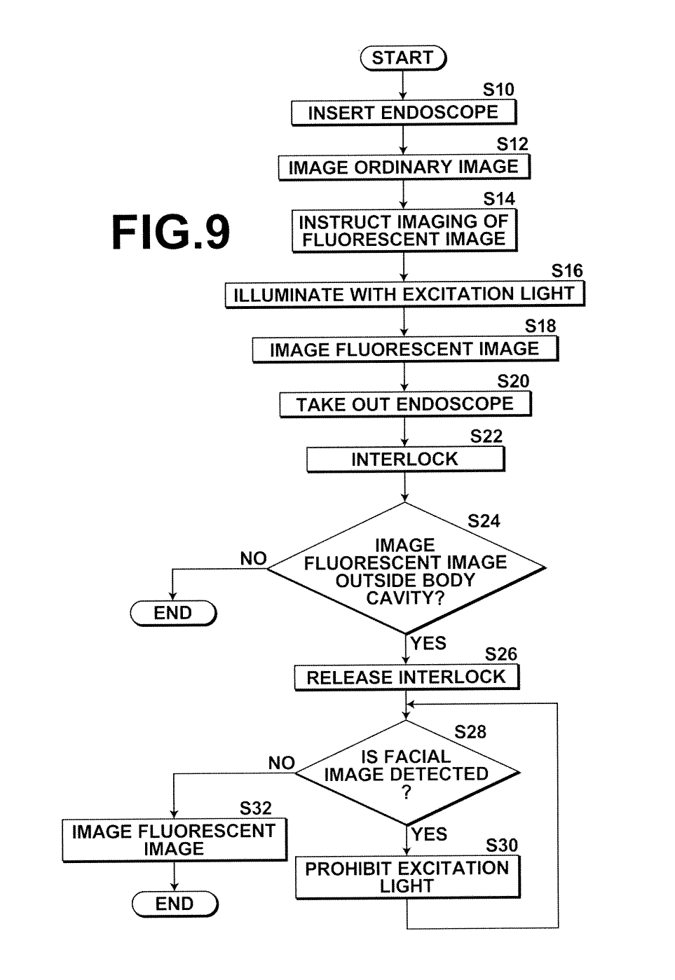

Next, with reference to a flow chart illustrated in FIG. 9, the action of the rigid endoscope system according to the present embodiment will be described.

First, the body cavity insertion unit 30 is inserted into a body cavity of a patient to be examined, and the leading end of the body cavity insertion unit 30 is placed in the vicinity of a region to be examined of the patient (step S10). Further, an ordinary image is imaged, and displayed (step S12)

Specifically, blue light output from the blue LID light source 40 in the light source apparatus 2 enters both of the light cables LC1 and LC2 simultaneously through the condensing lens 41 and the optical fiber splitter 42. Further, the blue light is guided by the light cables LC1 and LC2, and enters the body cavity insertion unit 30. Further, the blue light is guided by the multi-mode optical fibers 71 in the white light projection units 70 in the body cavity insertion unit 30. Further, the blue light is output from output ends of the multi-mode optical fibers, and a part of the blue light passes through the phosphors 72 to illuminate the region to be observed. The wavelength of the remaining part of the blue light, which has not passed through the phosphors 72, is converted to the wavelength of visible light of green through yellow by the phosphors 72, and the visible light of green through yellow illuminates the region to be observed. Specifically, the region to be observed is illuminated with white light composed of the blue light and the visible light of green through yellow.

Further, an ordinary image reflected from the region to be observed by illumination with white light enters the insertion member 30b through the imaging lens 30d provided at the leading end 30Y of the insertion member 30b. Further, the ordinary image is guided by a group of lenses in the insertion member 30b, and output toward the imaging unit 20.

When the ordinary image enters the imaging unit 20, the ordinary image is reflected in a right-angle direction by the dichroic prism 21. Further, the second image formation optical system 25 forms an image of the ordinary image on the imaging plane of the imaging device 26. The imaging device 26 images the ordinary image.

Further, image signals of R, G and B are output from the imaging device 26. After CDS/AGC (correlated double sampling/automatic gain control) processing and A/D (analog to digital) conversion processing are performed on the image signals at the imaging control unit 27, the image signals are output to the processor 3 through a cable 5.

After the ordinary image signals input to the processor 3 are temporarily stored in the ordinary image input controller 31, the ordinary image signals are stored in the memory 34. Further, the image processing unit 33 performs gradation correction processing and sharpness correction processing on ordinary image signals for each frame that have been read out from the memory 34. The ordinary image signals are sequentially output to the video output unit 35 after processing.

Further, the video output unit 35 generates display control signals by performing predetermined processing on the input ordinary image signals, and sequentially outputs display control signals for each frame to the monitor 4. The monitor 4 displays an ordinary image based on the input display control signals.

In the state in which an ordinary image is displayed as described above, for example, if a cancer or the like is found, and a doctor needs to excise a lymph node into which lymph flows from the vicinity of the cancer to perform pathological examination, ICG is administered to the vicinity of the cancer. Further, a fluorescent image of ICG is imaged, and displayed. At this time, imaging of the ordinary image may be ended, or continued.

Specifically, first, an instruction to start illumination with near-infrared light is input by using the operation unit 36 (step S14). The control unit 38 outputs control signals for starting output of near-infrared light to the LD drivers 46, 49 through the TG 37. The control unit 38 outputs the control signals based on the instruction to start illumination with near-infrared light, which has been input from the operation unit 36, and the LD drivers 46, 49 drive the near-infrared LD light sources 44, 47, respectively. The LD drivers 46, 49 make the near-infrared LD light sources 44, 47 output near-infrared light based on the control signals.

The near-infrared light output from the near-infrared LD light sources 44, 47 in the light source apparatus 2 enters the light cables LC3, LC4 through the condensing lenses 45, 48, respectively. Further, the near-infrared light enters the body cavity insertion unit 30 through the light cables LC3, LC4. Further, the near-infrared light is guided by the multi-mode optical fibers 61 in the near-infrared light projection units 60 in the body cavity insertion unit 30 to illuminate the region to be observed (step S16).

Further, an ICG fluorescent image that has been output from the region to be observed by illumination with excitation light of near-infrared light enters the insertion member through the imaging lens 30d provided at the leading end 30Y of the insertion member 30b. The ICG fluorescent image is guided by a group of lenses in the insertion member 30b, and output toward the imaging unit 20.

The ICG fluorescent image enters the imaging unit 20. After the ICG fluorescent image passes through the dichroic prism 21 and the near-infrared light cut filter 22, the ICG fluorescent image is formed on the imaging plane of the high-sensitivity imaging device 24 by the first image formation optical system 23, and the high-sensitivity imaging device 24 images the ICG fluorescent image. ICG fluorescent image signals are output from the high-sensitivity imaging device 24. After CDS/AGC (correlated double sampling/automatic gain control) processing and A/D conversion processing are performed on the ICG fluorescent image signals by the imaging control unit 27, the ICG fluorescent image signals are output to the processor 3 through the cable 5.

After the fluorescent image signals input to the processor 3 are temporally stored in the fluorescent image controller 32, the fluorescent image signals are stored in the memory 34. Fluorescent image signals for each frame are readout from the memory 34. After the image processing unit 33 performs predetermined image processing on the fluorescent image signals for each frame, the fluorescent image signals are sequentially output to the video output unit 35.

Further, the video output unit 35 performs predetermined processing on the input fluorescent image signals to generate display control signals. The video output unit 35 sequentially outputs display control signals for each frame to the monitor 4. The monitor 4 displays a fluorescent image based on the display control signals (step S18).

In the state in which a fluorescent image is displayed, a doctor excises a desirable lymph node, and takes out the lymph node from the patient's body, and the procedure ends. Then, an instruction to stop illumination with near-infrared light is received by the operation unit 36. The control unit 38 outputs a control signal to the light source apparatus 2 based on the instruction to stop illumination, and output of near-infrared light is stopped. After output of the near-infrared light has been stopped, the body cavity insertion unit 30 is taken out from the body cavity (step S20).

After the body cavity insertion unit 30 has been taken out from the body cavity, if the facial information detection unit 50 detects facial information, the detection result is output to the interlock unit 39. The interlock unit 39 outputs a control signal to the light source apparatus 2 through the control unit 38. After then, illumination with near-infrared light is prohibited until an instruction to release interlock is given (step S22). When the body cavity insertion unit 30 is taken out from the body cavity, if an instruction to stop illumination with near-infrared light is not given, the interlock unit 39 outputs, based on detection of facial information, a control signal to the light source apparatus 2 through the control unit 38. The interlock unit 39 prohibits output of near-infrared light after stopping output of near-infrared light.

When a lymph node that has been taken out from a body cavity is provided for pathological examination, as described above, it is desirable that a portion of the lymph node to which the cancer has metastasized is cut so that the metastasis portion is easily examined. In some cases, a user wants to observe a fluorescent image of the lymph node by illuminating the lymph node with near-infrared light again to clearly recognize the metastasis portion.

When a fluorescent image is imaged outside a patient's body as described above (step S24, YES), first, a release instruction to release interlock is given at the operation unit 36. A releasing-interlock instruction signal received by the operation unit 36 is output to the control unit 38. The control unit 38 releases, based on the releasing-interlock instruction signal, interlock by the interlock unit 39 (step S26).

When the control unit 38 receives the releasing-interlock instruction signal, the control unit 38 monitors the facial information detection result by the facial information detection unit 50 from the time when interlock has been released.

Further, an instruction to start illumination with near-infrared light is input by using the operation unit 36 again, and a fluorescent image is imaged by illumination with the near-infrared light based on the instruction to start illumination. At this time, the control unit 38 checks whether facial information is being detected by the facial information detection unit 50. If facial information is not being detected, the control unit 38 starts illumination with near-infrared light based on the instruction to start illumination with near-infrared light, and images a fluorescent image (step S28, NO).

In contrast, if facial information is being detected by the facial information detection unit 50, the control unit 38 controls the near-infrared LD light sources 44, 47 so that they do not output near-infrared light even if an instruction to start illumination with near-infrared light is given (step S30).

In the descriptions of the above embodiments, the control unit 38 monitors the facial information detection result by the facial information detection unit 50 from the time when the releasing-interlock instruction signal has been received. Alternatively, the control unit 38 may monitor the facial information detection result from the time when an instruction to start illumination with near-infrared light has been received at the operation unit 36, instead of from the time when the releasing-interlock instruction signal has been received. Then, if facial information is not being detected, the control unit 38 may control the near-infrared LD light sources 44, 47 so that they output near-infrared light based on the instruction to start illumination with near-infrared light. If facial information is being detected, the control unit 38 may control the near-infrared LD light sources 44, 47 so that they do not output near-infrared light.

In the aforementioned embodiments, the interlock unit 39 is provided. The interlock unit 39 prohibits illumination with near-infrared light when the facial information detection unit 50 has detected the facial information after the body cavity insertion unit 30 was taken out from the body cavity. However, it is not necessary that the interlock unit 39 is provided. When the interlock unit 39 is not provided, the control unit 38 monitors the facial information detection result by the facial information detection unit 50 constantly.

For example, after a procedure by a doctor ends, when the endoscope insertion unit 30 is taken out from a body cavity by mistake without giving an instruction to stop illumination with near-infrared light at the operation unit 36, if the facial information detection unit 50 detects facial information, the detection result is output to the control unit 38.

The control unit 38 outputs a control signal to the light source apparatus 2 based on the facial information detection result. The control unit 38 controls the light source apparatus 2 so that near-infrared light is not output while facial information is being detected by the facial information detection unit 50. The control unit 38 controls the light source apparatus 2 so that near-infrared light is output based on the instruction to start illumination with near-infrared light only when facial information is not being detected.

Further, output of near-infrared light may be controlled not only when the body cavity insertion unit 30 has been taken out from a patient's body after a procedure by a doctor ended, but also before the body cavity insertion unit 30 is inserted into the body cavity. Specifically, when an operation for outputting near-infrared light is checked, or calibration of near-infrared light is performed before the body cavity insertion unit 30 is inserted to the body cavity, an ordinary image may be imaged, and the facial information detection unit 50 may detect facial information. Further, output of near-infrared light may be controlled in such a manner that near-infrared light is not output while facial information is being detected by the facial information detection unit 50. Near-infrared light may be output based on an instruction to start illumination with near-infrared light only when facial information is not being detected.

In the aforementioned embodiments, it is desirable that facial information detection by the facial information detection unit 50 and monitoring by the interlock unit 39 and the control unit 38 are performed at a frequency of once per 0.25 second (one operation/0.25 second) or less frequently. When the frequency is set in such a manner, a load on the control system is low, and safe application of the technique is possible.

In the aforementioned embodiments, the image imaging apparatus (fluorescent endoscopy apparatus) of the present invention is applied to a rigid endoscope system. However, it is not necessary that the present invention is applied to the rigid endoscope system. For example, the present invention may be applied to a different kind of endoscope system including a flexible endoscopy apparatus. Further, it is not necessary that the present invention is applied to an endoscope system. The present invention may be applied to a so-called video-camera-type medical image imaging apparatus that does not include an insertion unit to be inserted into a patient's body.

What is claimed is:

1. A fluorescent endoscopy apparatus comprising:
an endoscope insertion unit that is inserted into a body cavity and that guides excitation light to illuminate a region to be observed;
an imaging unit that images a fluorescent image by receiving fluorescence that has been output from the region to be observed by illumination with the excitation light and guided by the endoscope insertion unit;
a facial information detection unit that detects facial information about a person in an imaging image that has been imaged by the imaging unit by receiving light guided by the endoscope insertion unit; and
an interlock unit that prohibits illumination with the excitation light when the facial information detection unit has detected the facial information.

2. A fluorescent endoscopy apparatus, as defined in claim 1, further comprising:
a canceling-prohibition unit that cancels prohibition of illumination with the excitation light after the illumination has been prohibited by the interlock unit; and
an excitation light illumination control unit that checks the result of detecting the facial information from the time when the prohibition of illumination has been canceled by the canceling-prohibition unit, and that controls illumination with the excitation light so that the illumination is not performed while the facial information is being detected by the facial information detection unit.

3. A fluorescent endoscopy apparatus, as defined in claim 1, further comprising:
a canceling-prohibition unit that cancels prohibition of illumination with the excitation light after the illumination has been prohibited by the interlock unit;
a starting-illumination instruction receiving unit that receives an instruction to start illumination with the excitation light after the prohibition of illumination has been canceled by the canceling-prohibition unit; and
an excitation light illumination control unit that checks the result of detecting the facial information from the time when the instruction to start illumination has been received, and that controls illumination with the excitation light so that the illumination is not performed while the facial information is being detected by the facial information detection unit.

4. A fluorescent endoscopy apparatus comprising:
an endoscope insertion unit that is inserted into a body cavity and that guides excitation light to illuminate a region to be observed;
an imaging unit that images a fluorescent image by receiving fluorescence that has been output from the region to be observed by illumination with the excitation light and guided by the endoscope insertion unit;
a facial information detection unit that detects facial information about a person in an imaging image that has been imaged by the imaging unit by receiving light guided by the endoscope insertion unit; and
an excitation light illumination control unit that controls illumination with the excitation light so that the illumination is not performed while the facial information is being detected by the facial information detection unit.

5. A fluorescent endoscopy apparatus, as defined in claim 1, wherein the facial information detection unit detects a round flesh-color portion in the imaging image, and detects the round flesh-color portion as the facial information when the ratio of the area of the round flesh-color portion to the area of the imaging image is greater than or equal to a predetermined threshold value.

6. A fluorescent endoscopy apparatus, as defined in claim 4, wherein the facial information detection unit detects a round flesh-color portion in the imaging image, and detects the round flesh-color portion as the facial information when the ratio of the area of the round flesh-color portion to the area of the imaging image is greater than or equal to a predetermined threshold value.
